(19) United States
(12) Patent Application Publication
(10) Pub. No.: US 2013/0245564 A1
(43) Pub. Date: Sep. 19, 2013

(54) SAFETY DEVICE FOR A SYRINGE

(71) Applicant: Ren-Yue Cheng, Taipei City (TW)

(72) Inventor: Ren-Yue Cheng, Taipei City (TW)

(21) Appl. No.: 13/588,027

(22) Filed: Aug. 17, 2012

(30) Foreign Application Priority Data
Mar. 15, 2012 (TW) 101108880

(51) Int. Cl. A61M 5/32

(52) U.S. Cl. 604/195

(57) ABSTRACT

A safety device is adapted to be sleeved on a needle unit of a syringe. The safety device includes a barrel unit movable with respect to a base seat of the needle unit and having a connecting end portion and a flexible end portion that has a pair of coupling sections provided respectively with first and second engaging members. The flexible end portion is operable between an opened state, where the coupling sections define an opening that permits extension of a cannula of the needle unit when the barrel unit is in a retracted state, and a closed state, where the flexible end portion deforms to result in engagement between the first and second engaging members to thereby close the opening when the barrel unit is moved to an extended state.

### CROSS-REFERENCE TO RELATED APPLICATION

This application claims priority of Taiwanese Patent Application No. 101108880, filed on Mar. 15, 2012.

### BACKGROUND OF THE INVENTION

1. Field of the Invention

This invention relates to a safety device for a syringe.

2. Description of the Related Art

As shown in FIG. 1, a conventional syringe 1 includes a cylinder body 11, a needle unit 12 and a cover 13 for covering the needle unit 12. The needle unit 12 includes a base seat 121 connected to the cylinder body 11, and a cannula 122 extending from the base seat 121. The cover 13 is elongated and is made of plastic.

The cover 13 is tightly fitted on the base unit 121 to enclose the cannula 122 therein to prevent the cannula 122 from wounding a medical personnel and being contaminated due to exposure to the air, and would be removed from the base unit 121 when the conventional syringe 1 is in use. After use of the conventional syringe 1, the cover 13 has to be replaced to cover the cannula 122 before disposal the syringe 1.

However, the medical personnel may be wounded by the cannula 122 due to misalignment of the cover 13 with the cannula 122 during the replacement of the cover 13.

### SUMMARY OF THE INVENTION

Therefore, the object of the present invention is to provide a safety device for a syringe to overcome the aforesaid drawback of the prior art.

According to this invention, the safety device is adapted to be sleeved on a needle unit of a syringe. The needle unit includes a base seat and a cannula that extends from the base seat along an axis. The safety device includes a barrel unit adapted to be movable along the axis with respect to the base seat of the needle unit of the syringe between an extended state, where the cannula is disposed within the barrel unit, and a retracted state, where the cannula extends outwardly of the barrel unit. The barrel unit has a connecting end portion that is adapted to be connected to the base seat, and a flexible end portion that is adapted to be distant from the base seat, that has a pair of coupling sections provided respectively with first and second engaging members, said flexible end portion being operable between an opened state, where the coupling sections define an opening that permits extension of the cannula when the barrel unit is at the retracted state, and a closed state, where the flexible end portion deforms to result in engagement between the first and second engaging members to thereby close the opening when the barrel unit is at the extended state.

### BRIEF DESCRIPTION OF THE DRAWINGS

Other features and advantages of the present invention will become apparent in the following detailed description of the preferred embodiment of this invention, with reference to the accompanying drawings, in which:

FIG. 1 is a fragmentary partly exploded perspective view of a conventional syringe;

FIG. 2 is an exploded perspective view of a preferred embodiment of a safety device for a syringe according to this invention;

FIG. 3 is an assembled perspective view of the preferred embodiment, illustrating a barrel unit at an extended state and coupling sections of a flexible end portion of the barrel unit at an opened state;

FIG. 4 is a sectional view of the preferred embodiment, illustrating the barrel unit at the extended state and the coupling sections of the flexible end portion of the barrel unit at the opened state;

FIG. 5 is another sectional view of the preferred embodiment, illustrating the barrel unit at a retracted state;

FIG. 6 is another assembled perspective view of the preferred embodiment, illustrating the barrel unit at the extended state and the coupling sections of the flexible end portion of the barrel unit at a closed state;

FIG. 7 is a fragmentary sectional view of the preferred embodiment, illustrating the coupling sections of the flexible end portion of the barrel unit at the closed state; and

FIG. 8 is another fragmentary sectional view of a modification of the coupling sections of the flexible end portion of the barrel unit.

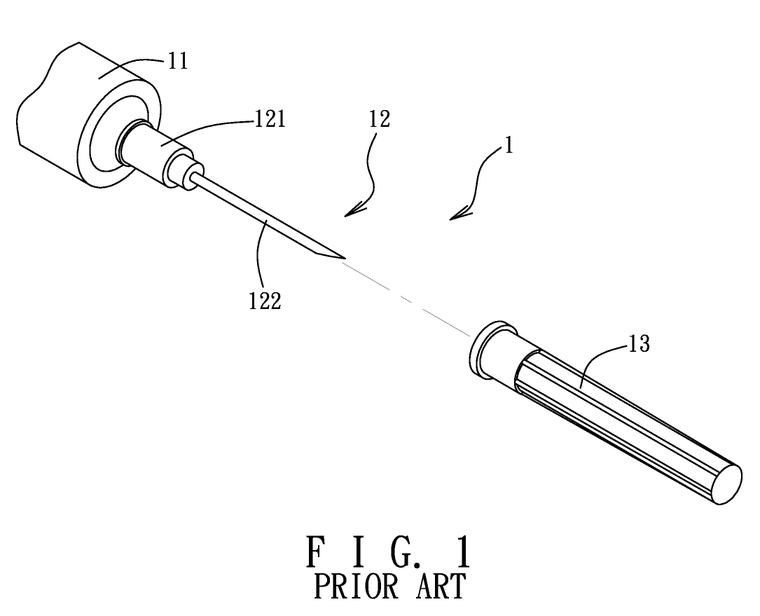

### DETAILED DESCRIPTION OF THE PREFERRED EMBODIMENT

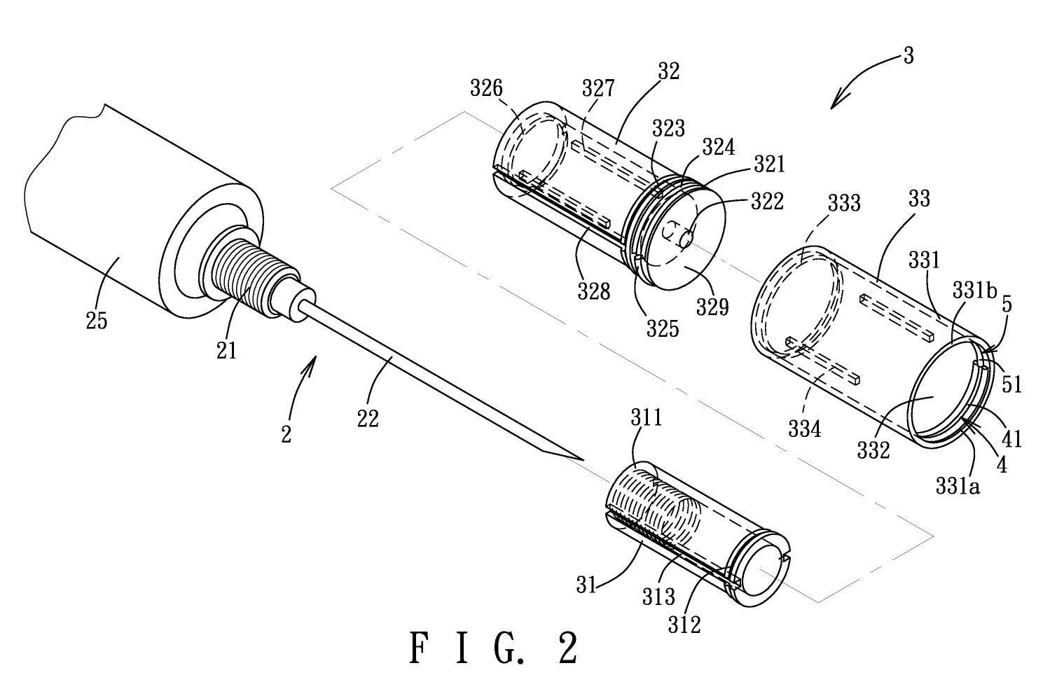

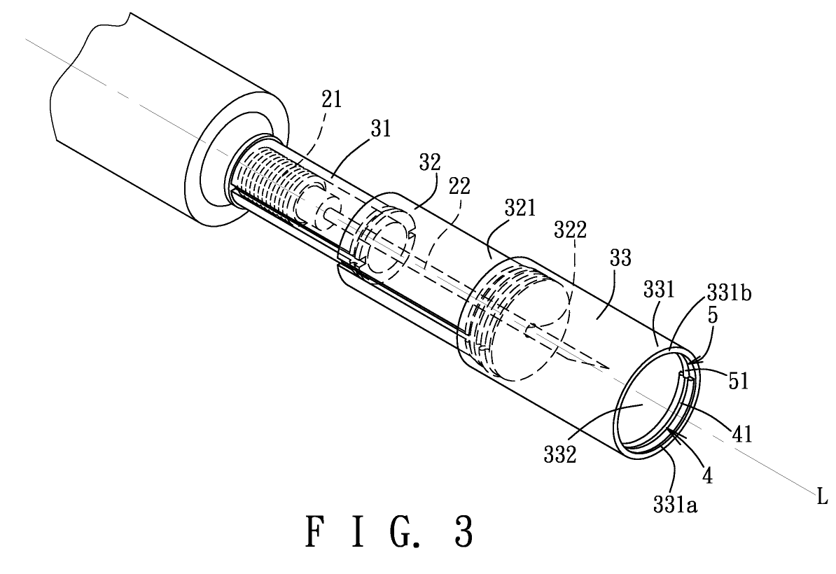

As shown in FIGS. 2 and 3, a preferred embodiment of a safety device according to this invention is adapted to be sleeved on a needle unit 2 of a syringe. The needle unit 2 includes a base seat 21 and a cannula 22 that extends from the base seat 21 along an axis (L).

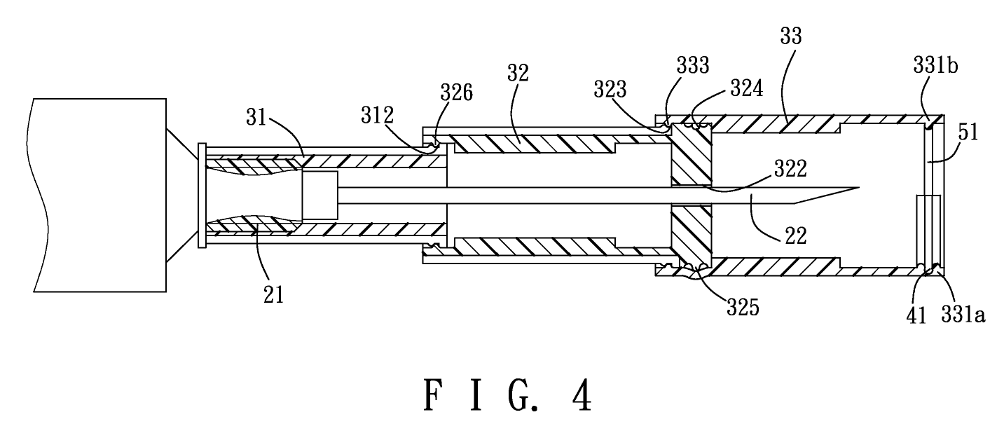

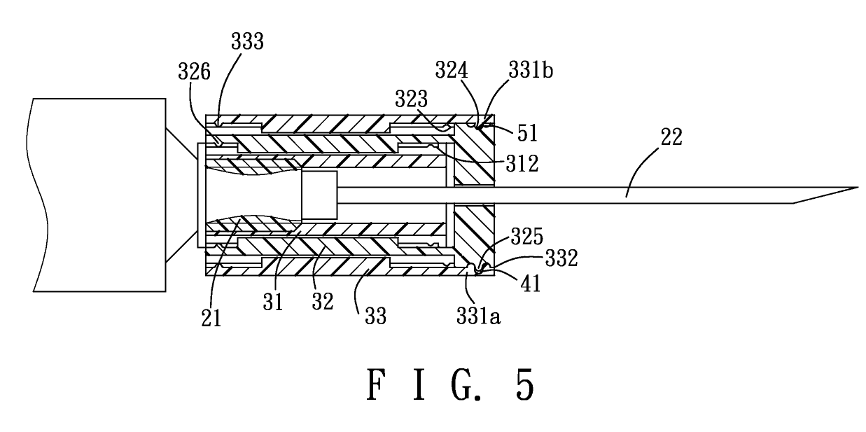

The safety device comprises a barrel unit 3 that is movable along the axis (L) with respect to the base seat 21 between an extended state (see FIGS. 3 and 4), where the cannula 22 is disposed within the barrel unit 3, and a retracted state (see FIG. 5), where the cannula 22 extends outwardly of the barrel unit 3. The barrel unit 3 includes a base barrel 31, an intermediate barrel 32 and a terminal barrel 33. The base barrel 31 has a connecting end portion 311 that is adapted to be connected threadedly to the base seat 21 of the needle unit 2. It should be noted that the connecting end portion 311 may have a part sleeved fittingly on the base seat 21, or may be connected directly to a syringe body 25 to which the needle unit 2 is coupled.

The intermediate barrel 32 is sleeved slidably on the base barrel 31 and has a positioning end portion 321 adapted to be distant from the base seat 21 of the needle unit 2. The positioning end portion 321 of the intermediate barrel 32 has an end wall 329 that is normal to the axis (L) and that is formed with a positioning hole 322 adapted for extension of the cannula 22 of the needle unit 2 during the movement of the barrel unit 3 between the retracted and extended states.

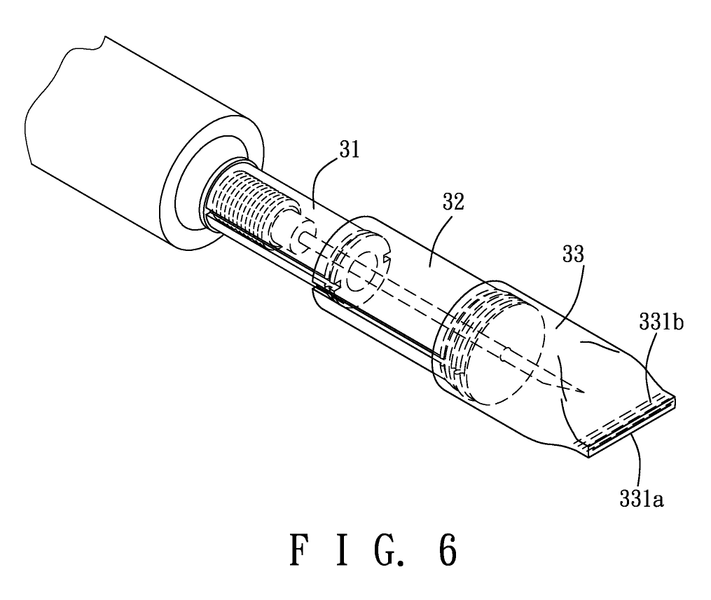

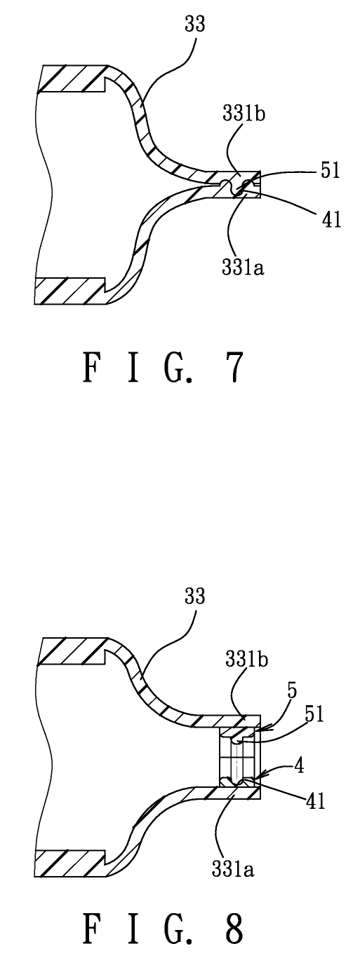

The terminal barrel 33 is sleeved slidably on the intermediate barrel 32 and has a flexible end portion 331 that is adapted to be distant from the base seat 21. The flexible end portion 331 has a pair of coupling sections 331a, 331b provided respectively with first and second engaging members 4, 5, and is operable between an opened state (see FIGS. 3 to 5), where the coupling sections 331a, 331b define an opening 332 that permits extension of the cannula 22 when the barrel unit 3 is in the retracted state, and a closed state (see FIGS. 6 and 7), where the flexible end portion 331 deforms to result in engagement between the first and second engaging members 4, 5 to thereby close the opening when the barrel unit 3 is in the extended state.

In this embodiment, the coupling sections 331a, 331b are integrally and respectively formed with the first and second engaging members 4, 5. The first engaging member 4 is a first coupling groove 41, and the second engaging member 5 is a first coupling protrusion 51. In other embodiments of this invention, as shown in FIG. 8, the first and second engaging members 4, 5 may be adhered on the coupling sections 331a, 331b, while the first engaging member 4 is formed with the first coupling groove 41, and the second engaging member 5 is formed with the first coupling protrusion 51. Moreover, the first and second engaging members 4, 5 may be configured as soft magnets.

Each of the base barrel 31 and the intermediate barrel 32 has an outer surface formed with a pair of guiding grooves 313, 328 extending in the direction of the axis (L). Each of the intermediate barrel 32 and the terminal barrel 33 has an inner surface formed with a pair of guiding protrusions 327, 334 that engage slidably and respectively the corresponding guiding grooves 313, 328 of a respective one of the base barrel 31 and the intermediate barrel 32. The positioning end portion 321 of the intermediate barrel 32 is formed with a second coupling groove 324 and a second coupling protrusion 325.

The outer surface of each of the base barrel 31 and the intermediate barrel 32 is formed with an annular positioning groove 312, 323 at a position distal from the base seat 21. The inner surface of each of the intermediate barrel 32 and the terminal barrel 33 is formed with an annular positioning protrusion 326, 333 that is approximate to the base seat 21. Moreover, the second coupling groove 324 and the second coupling protrusion 325 of the intermediate barrel 32 are disposed between the positioning groove 323 and an end edge of the positioning end portion 321.

As shown in FIG. 4, when the barrel unit 3 is at the extended state, the positioning protrusion 326 of the intermediate barrel 32 engages removably the positioning groove 312 of the base barrel 31, while the positioning protrusion 333 of the terminal barrel 33 engages removably the positioning groove 323 of the intermediate barrel 32, thereby positioning the base barrel 31, the intermediate barrel 32 and the terminal barrel 33 with one another. Further referring to FIG. 5, in use of the syringe, the terminal barrel 33 is moved toward the base seat 21 to move the barrel unit 3 telescopically from the extended state to the retracted state. During the movement of the barrel unit 3 from the extended state to the retracted state, the terminal barrel 33 and the intermediate barrel 32 can move stably without rotation about the axis (L) by virtue of the engagements between the guiding protrusions 327, 334 and the guiding grooves 313, 328. When the barrel unit 3 is in the retracted state, the second coupling groove 324 and the second coupling protrusion 325 of the intermediate barrel 32 are engaged respectively with the first coupling protrusion 51 and the first coupling groove 41 of the base barrel 31. After use, the barrel unit 3 is moved from the retracted state to the extended state, and the flexible end portion 331 of the terminal barrel 33 is squeezed from the opened state to the closed state before disposal of the syringe.

To sum up, the barrel unit 3 can effectively protect a medical personnel from being wounded by the cannula 22, and is not required to be removed during use of the syringe.

While the present invention has been described in connection with what is considered the most practical and preferred embodiment, it is understood that this invention is not limited to the disclosed embodiment but is intended to cover various arrangements included within the spirit and scope of the broadest interpretation and equivalent arrangements.

What is claimed is:

1. A safety device adapted to be sleeved on a needle unit of a syringe, the needle unit including a base seat and a cannula that extends from the base seat along an axis, said safety device comprising a barrel unit that is adapted to be movable along the axis with respect to the base seat of the needle unit of the syringe between an extended state, where the cannula is disposed within said barrel unit, and a retracted state, where the cannula extends outwardly of said barrel unit, said barrel unit having a connecting end portion that is adapted to be connected to the base seat, and a flexible end portion that is adapted to be distant from the base seat, and that has a pair of coupling sections provided respectively with first and second engaging members, said flexible end portion being operable between an opened state, where said coupling sections define an opening that permits extension of the cannula when said barrel unit is at the retracted state, and a closed state, where said flexible end portion deforms to result in engagement between said first and second engaging members to thereby close said opening when said barrel unit is at the extended state.

2. The safety device as claimed in claim 1, wherein said first engaging member of said coupling section is a first coupling groove, and said second engaging member of said coupling section is a first coupling protrusion.

3. The safety device as claimed in claim 1, wherein said first and second engaging members are adhered respectively on said coupling sections, and are formed respectively with a first coupling groove and a first coupling protrusion.

4. The safety device as claimed in claim 1, wherein said barrel unit includes abase barrel having said connecting end portion, an intermediate barrel sleeved slidably on said base barrel, and a terminal barrel sleeved slidably on said intermediate barrel and having said flexible end portion.

5. The safety device as claimed in claim 4, wherein said first and second engaging members of said coupling sections are formed respectively as a first coupling groove and a first coupling protrusion.

6. The safety device as claimed in claim 5, wherein said intermediate barrel has a positioning end portion adapted to be distant from the base seat of the needle unit, and formed with a second coupling protrusion and a second coupling groove that are engaged respectively with said first coupling groove and said first coupling protrusion when said barrel unit is at the retracted state.

7. The safety device as claimed in claim 6, wherein said positioning end portion of said intermediate barrel has an end wall that is normal to the axis and that is formed with a positioning hole adapted for extension of the cannula of the needle unit during the movement of said barrel unit between the retracted and extended states.

8. The safety device as claimed in claim 1, wherein said connecting end portion of said barrel unit is adapted to be connected threadedly to the base seat of the needle unit.
